(19) United States
(12) Patent Application Publication
(10) Pub. No.: US 2021/0327578 A1
(43) Pub. Date: Oct. 21, 2021

(54) System and Method for Medical Triage Through Deep Q-Learning

(71) Applicant: Babylon Partners Limited, London (GB)

(72) Inventors: Albert James Thomas Buchard, London (GB); Baptiste Bouvier, London (GB); Michail Livieratos, London (GB); Rory Beard, London (GB); Kostis Gourgoulias, London (GB); Saurabh Johri, London (GB); Giulia Prando, London (GB)

(21) Appl. No.: 16/849,162

(22) Filed: Apr. 15, 2020

(30) Foreign Application Priority Data
Apr. 8, 2020 (GR) 20200100179

(51) Int. Cl. G16H 50/20; G06N 3/08

(52) U.S. Cl. CPC G16H 50/20; G06N 3/08

(57) ABSTRACT

The present application presents a methodology that applies reinforcement learning to train a neural network to perform medical triage by analyzing medical evidence of a patient (for instance, obtained via an interface with the patient), assigning a triage level to the patient where sufficient evidence has been obtained to make a reliable triage decision, and requesting more evidence where needed. By learning when to ask for more information and when to make a decision, the neural network can be trained to make quicker decisions on fewer pieces of evidence whilst still ensuring an accurate and safe triage level is determined.

### TECHNICAL FIELD

The present disclosure relates to systems and methods for determining a triage level for a patient and for training a neural network to determine a triage level for a patient. In particular, but without limitation, this disclosure relates to training neural networks through reinforcement learning to perform medical triage classification based on observations (e.g. symptoms and risk factors) presented by patients.

### BACKGROUND

Medical triage is the process of determining the urgency by which patients need to be treated or reviewed by a medical professional based on the severity of their condition.

In general, medical triage assigns a relative priority to each patient based on the symptoms being displayed.

Be it through telephone interaction or performed face to face by a trained healthcare professional; the triage process aims to uncover enough medical evidence to make an informed decision about the appropriate point of care given a patient's presentation.

Medical triage is of paramount importance to healthcare systems, allowing for the correct orientation of patients and allocation of the necessary resources to treat them adequately.

While strong decision-tree methods exist to triage patients based on their presentation, those trees are not immediately applicable in a fully automated setting without early inference. Furthermore, while deep-learning approaches are able to classify patients based on image recognition, they are often only able to classify very specific conditions (e.g. skin cancer) and are unable to be applied to more broadly to triage based on general clinical signs (e.g. presented symptoms).

### SUMMARY

The present application presents a methodology that applies reinforcement learning to train a neural network to perform medical triage by analyzing medical evidence of a patient (for instance, obtained via an interface with the patient), assigning a triage level to the patient where sufficient evidence has been obtained to make a reliable triage decision, and requesting more evidence where needed. By learning when to ask for more information and when to make a decision, the neural network can be trained to make quicker decisions on fewer pieces of evidence whilst still ensuring an accurate triage level is determined.

The reinforcement learning can be performed on pre-curated and labelled sets of patient evidence relating to one or more observed attributes of a patient (for instance, symptoms and/or risk factors). The neural network can be provided subsets of the evidence piece by piece and trained to learn when to best make a triage decision and when to request more information (through obtaining additional evidence from the full set of evidence).

This allows the system to be trained to learn when to best make a triage decision whilst avoiding exposing real-life patients at risk.

The information regarding the attributes of the patient (the set of evidence) may be labelled with a set of predetermined triage actions for the patient (e.g. determined by a human expert). The probability that a given triage level is correct can be determined from this set of predetermined triage actions. Accordingly, the neural network can be trained using rewards determined from a probability that a given action is correct based on the set of predetermined triage actions. This allows the neural network to be trained effectively to make accurate triage decisions.

According to a first aspect there is provided a computer-implemented method for training a neural network to determine a triage level for a patient, the neural network being for controlling an agent to determine a triage class for one or more patients. The method comprises: requesting information regarding one or more observed attributes of a patient; receiving the information regarding the one or more observed attributes of the patient; and defining a state of the patient for an initial step, the state comprising the one or more observed attributes. The method further comprises, for one or more steps beginning at the initial step and continuing until either a triage action is selected or a maximum number of steps has been reached: inputting the state for the step into the neural network to determine an action in response to the input state, the action being selected from a plurality of actions including: an information request action, and a plurality of triage actions; obtaining a reward for each of the triage actions based on a probability of the corresponding triage action being correct that is determined based on a set of predetermined triage actions for the patient; and applying the selected action. Applying the selected action includes, in response to the information request action being selected requesting one or more additional observed attributes of the patient and, in response to receiving the one or more additional observed attributes: defining a next state for a next step to include the one or more observed attributes and the one or more additional observed attributes of the patient; defining an experience for the step comprising the state for the step, the selected action, a reward for each triage action of the predefined selection, and the next state; and moving to the next step. Applying the selection action further includes, in response to one of the triage actions being selected, assigning a corresponding triage level for the selected triage action to the patient. The method further comprises updating parameters of the neural network based on the defined experiences.

In light of the above, embodiments apply reinforcement learning with actions selected from a set of triage actions and an information request action. Importantly, the methodology described herein includes a counterfactual reward wherein each experience includes a reward for each possible triage action, even if the triage action is not selected at that step. This is possible as each triage action is terminal (it results in a triage level being assigned to the patient), so can be easily incorporated into the training of the system even if an information request action is selected.

The methods described herein may utilize deep Q-learning wherein a neural network is configured to determined state-action values (Q-values) that are used to dictate the actions by the agent (e.g. through selection of the action corresponding to a maximum Q-value). As each reward for each triage action corresponds to a probability based on a predetermined set of triage actions, the rewards for each triage action may be calculated by the method either before the training or during the running of the training.

According to an embodiment, the neural network is configured to determine state-action values for each action based on input states and the parameters of the neural network. The actions are determined based on the state-action values for the actions. Furthermore, updating parameters of the neural network based on the defined experiences comprises selecting a set of one or more experiences from the defined experiences. Updating parameters of the neural network further comprises, for each experience in the set of one or more experiences: determining, for each of the plurality of actions, a state-action value for the state in the experience using the neural network; calculating, for each of the plurality of actions, a target state-action value for the state in the experience; and determining, for each of the plurality of actions, a difference between the corresponding state-action value and the corresponding target state-action value. The parameters of the neural network are updated based on each determined difference.

The updating of the parameters may be to reduce the difference between the state-action values and the target state-action values. This may be based on optimizing an objective function, e.g. to minimize the difference. Importantly, the state action values are determined for each of the plurality of actions, and not simply the action that has been selected.

According to an embodiment, calculating, for each of the plurality of actions, a target state-action value for the state in the experience comprises: determining the target state-action value for each of the triage actions based on the reward for the corresponding triage action; and determining the target state-action value for the information request action based on the target state-action values for the triage actions.

According to a further embodiment, the target state-action value for the information request is determined based on a maximum state-action value of the state-action values either for the triage actions or for a predetermined selection of the triage actions. This allows the reward for an information request action to be determined based on the probability that the agent would select a correct triage decision (as the agent may be configured to select the action that has the maximum state-action value) or, conversely, the probability that the agent would select an incorrect triage decision. Accordingly, the target state action value (target Q-value) for the ask action represents the results of a specific probabilistic query which encodes a particular choice for a stopping criterion.

The predetermined selection of triage actions may be determined based on the predetermined triage actions for the patient, e.g., may be a selection of triage actions that have a sufficient probability of being correct based on the predetermined triage actions. This may be based on the number of times the corresponding triage action appear within the predetermined triage actions and/or a range of triage levels that are deemed correct (e.g. safe or appropriate) based on the predetermined triage actions. A variety of metrics may be used when determine the predefined triage actions that may be deemed to be correct. For instance, appropriateness or safety may be judged based on a distribution of the correct triage actions in the predefined triage actions. Alternatively, a classifier or other model may be applied to the evidence, having been trained based on a predefined set of correct triage levels.

According to an embodiment, determining the target state-action value for the information request comprises determining a probability that the maximum state-action value for the state relates to a triage action that is incorrect given the state. This can be considered to be equivalent to determining a probability that the maximum state-action value for the state relates to a triage action.

A triage action may be incorrect if the neural network is not predicting a correct triage level (as defined by the predetermined triage actions) to a sufficient degree of confidence (e.g. not assigning a sufficiently high state-action value to a triage action that is likely to be correct). A correct triage level may be one that is deemed safe and/or correct based on the predetermined triage actions.

A safe triage level may be any level that is above a minimum triage level from the triage levels associated with the predetermined triage actions. An acceptable triage action might be a triage level that is within the range of triage levels including and between a maximum and minimum triage level within the triage levels corresponding to the predetermined triage actions.

According to an embodiment, determining the target state-action value for the information request comprises determining a probability that either the maximum state-action value for the state relates to a triage action that is incorrect given the state or that a maximum state-action value for the next state relates to a triage action that is correct given the next state.

According to an embodiment, determining the target state-action value for the information request comprises determining a probability that a maximum state-action value for a subsequent state relates to a triage action that would be correct given the subsequent state and that the maximum state-action value for each state before the subsequent state relates to a triage action that is incorrect given the corresponding state.

According to an embodiment, the method comprises determining the reward for each triage action, including calculating, for each triage action, a probability that the corresponding triage action is correct based on the set of predetermined triage actions for the corresponding patient.

According to an embodiment, the probability that the corresponding triage action is correct is determined based on a number of times the corresponding triage action appears in the set of predetermined triage actions for the corresponding patient. This can be a normalized probability.

According to an embodiment, applying the neural network comprises obtaining a set of observed attributes of the patient, wherein defining the state includes selecting a subset of the set of observed attributes of the patient, and the one or more additional observed attributes are obtained through selection from the set of observed attributes.

Accordingly, the training can be run on a set of predetermined observed attributes, with only a subset (e.g. one or more) of observed attributes being provided to the neural network at each step. The one or more additional observed attributes may be hitherto unselected attributes.

According to an embodiment, the neural network is configured to determine state-action values for each action based on input states and the parameters of the neural network. The actions are determined based on the state-action values for the actions. Noise is added to the state-action value for the information request action prior the determination of the action. This encourages exploration during training with regard to the information request action. Noise may only be added to the state-action value for the information request action (i.e. noise need not be added to the triage actions). The goal of exploration in this case is to evaluate when to stop rather than to gather information about specific triage rewards. The noise may decay over the number of steps. For instance, the noise may be determined based on a Gaussian function with a standard deviation that decreases as the number of steps increases.

According to a further aspect there is provided a computer-implemented method for determining a triage level for a patient including: obtaining a neural network trained according to one of the above methods; requesting information regarding one or more observed attributes of the patient; receiving the information regarding the one or more observed attributes of the patient; and defining a state of the patient for an initial step, the state comprising the one or more observed attributes. The method further comprises, for one or more steps beginning at the initial step and continuing until either a triage action is selected or a maximum number of steps has been reached: inputting the state for the step into the neural network to determine an action in response to the input state, the action being selected from a plurality of actions including: an information request action, and a plurality of triage actions; and applying the selected action. Applying the selected action includes, in response to the information request action being selected, requesting and receiving information regarding one or more additional observed attributes of the patient, defining a next state for a next step to include the one or more observed attributes and the one or more additional observed attributes of the patient and moving to the next step. Applying the selected action further includes, in response to one of the triage actions being selected, outputting the corresponding triage level for the selected triage action as the triage level for the patient.

Any of the methods described herein may be embodied in a system comprising a processor configured to perform the method. Equally, the any of the methods described herein may be embodied in a non-transitory computer readable medium having stored therein computer executable instructions that, when executed by a processor, cause the processor to perform the method.

### BRIEF DESCRIPTION OF THE DRAWINGS

Arrangements of the present invention will be understood and appreciated more fully from the following detailed description, made by way of example only and taken in conjunction with drawings in which:

FIG. 1 shows a schematic of a triage system according to an embodiment;

FIG. 2 shows a method of determining a triage level using an artificial agent trained according to an embodiment;

FIG. 3 shows a method of obtaining experiences for training a triage system according to an embodiment;

FIG. 4 shows a method of training a triage system according to an embodiment; and

FIG. 5 shows a computing system for implementing embodiments described herein.

### DETAILED DESCRIPTION

The present application presents a deep reinforcement learning approach for triaging patients. The methodology herein trains a neural network system to assign a triage level to a patient. Importantly, the methodology also trains the neural network to learn when insufficient information has been obtained from the patient so far and, in this case, to request additional information from a patient (e.g. through issuing a question to the patient). This allows the system to control the information gathering process, and allows the system to make accurate triage decisions based on fewer pieces of evidence. This is important as it speeds up the triage process, reducing the number of questions that the patient must answer before being assigned an appropriate triage level.

For many patients, medical triage is the first organized contact with the healthcare system. Be it through telephone interaction or performed face to face by a trained healthcare professional; the triage process aims to uncover enough medical evidence to make an informed decision about the appropriate point of care given a patient's presentation. The clinician's task is to plan the most efficient sequence of questions in order to make a fast and accurate triage decision.

Although internationally recognized systems exist, with clearly defined decision trees based on expert consensus, in practice, the nature of the triage task is not a passive recitation of a learned list of questions. Triage is an active process through which the clinician must make inferences about the causes of the patient's presentation and update their plan following each new piece of information.

To deploy triage systems in healthcare settings, a population of clinicians needs to undergo training to ensure the reliability and quality of their practice. No system seems to be superior overall, but their performance varies significantly across studies.

In order to improve patient safety and quality of care, many decision support tools are designed on top of those decision trees to standardize and automate the triage process with mixed results.

Deep learning approaches to clinical decision making exist with several applications in perceptual settings, in which the decision relies on image recognition and not on clinical signs. The production of automated triage systems that do not rely on expert-crafted decision trees, and are able to learn from data, is a more difficult task.

The embodiments described herein make use of reinforcement learning acting upon clinical vignettes, each describing a patient presentation in terms of symptoms and risk factors. The system learns when a triage decision can be made and when further information is required (and, as such, further questions are necessary).

Learning a triage system from a detailed distribution of patient trajectories allows for the correction of inherent biases of expert-crafted systems and allows the system to be tailored for a specific target population. Given sufficient training data, the embodiments described herein can be designed to minimize the risk for the patients by striking the right balance between information gathering and decision-making.

Reinforcement Learning

Reinforcement learning is a natural approach for problems requiring the optimization of sequences of actions in order to reach complex objectives. Although interaction with real patients is usually not ethically possible, it is possible to use observational and generated datasets to apply reinforcement learning approaches to the healthcare setting.

Reinforcement learning describes an approach to learning where an agent learns through interactions with an environment, gathering rewards and penalties for the actions performed. Under the paradigm of reinforcement learning, the general interaction between an agent and its environment is well defined.

The environment describes the world in which the agent is evolving in time.
The environment keeps track of the agent's state sₜ∈S, with S referring to the state-space, the set of all valid states the agent can be in.
The environment processes the agent's actions at aₜ∈ [symbol] at each time t, with [symbol] being the set of all possible actions (called the action-space of the agent).
The environment encodes the system dynamics, fully defined by the transition function p:S× [symbol] ×S→[0,1], which gives the probability P(s′|s,a) to transition to state s′ given that the agent did action a in state s:

p(s′|s,a)=P(sₜ₊₁=s′|sₜ=s,aₜ=a)
The environment also defines a notion of optimal behavior through a reward function r:S× [symbol] + [symbol] , here defined as a map from a state-action pair to a real value (e.g., +1 or a positive reward, and −1 for a penalty), which is returned at each time step to the agent.

An agent is an entity that performs actions into the environment, given its current state and a policy π:S× [symbol] →[0,1], a function that gives the probability of an action when in a particular state. The set of actions that an agent takes and the set of states that are consequently visited constitute a trajectory τ∈ [symbol] , denoted as r=(a₀, a₀, s₁, a₁, . . . ). The goal of training a reinforcement learning agent is to learn a function, called the optimal policy π*: S→ [symbol] that mas an agent's state to a specific action so that the reward received by the agent is maximized in expectation across all interactions.

Q-Learning

Model-free reinforcement learning methods describe settings where an agent does not have access to the dynamics of the environment and cannot interrogate or learn the transition function. Two main classes of model-free algorithms exist: a) policy-based methods, which aim to learn the policy directly; and, b) value-based methods, which aim to learn one or several value functions to guide the agent policy toward high reward trajectories. The embodiments described herein, make use a variant of Q-Learning, which is a model-free and value-based method, called Deep Q-Learning.

In Q-Learning, the agent does not learn a policy function directly but instead learns a proxy state-action value function Q(s, a). This function approximates an optimal function Q*(s,a) defined as the maximum expected return achievable by any policy a over all possible trajectories τ, given that in state s the agent performs action a and the rest of the trajectory τ is generated by following n, denoted as τ˜π (with a slight abuse of notation).

$${Q}^{*} ( s , a ) = {max}_{π} {𝔼}_{τ \sim π} [ R ( τ \vert {s}_{0} = s , {a}_{0} = a ) ]$$

with R(τ|s₀,a₀) being the return function defining the return from the rewards gathered over the trajectory τ:

$$R ( τ \vert {s}_{0} , {a}_{0} ) = r ( {s}_{0} , {a}_{0} ) + {\sum }_{{s}_{t} , {a}_{t} ∈ τ}^{∞} {γ}^{t} r ( {s}_{t} , {a}_{t} )$$

The return function is therefore a weighted sum of rewards over the trajectory τ. The weight γ∈[0,1] is called the discount factor. It encodes the notion that sequences of actions are usually finite, and one gives more weight to the current reward.

Q*(s,a) has an optimal substructure and can be written recursively using the Bellman Equation, which treats each decision step as a separate sub-problem:

$${Q}^{*} ( s , a ) = r ( s , a ) + γ {\sum }_{{s}^{′}} p ( {s}^{′} \vert s , a ) {max}_{{a}^{′}} {Q}^{*} ( {s}^{′} , {a}^{′} )$$

This function encodes the value of performing a particular action a when in state s as the sum of the immediate reward returned by the environment and the weighted expected rewards obtained over the future trajectory. The future trajectory being generated by a greedy policy that selects the actions that maximize Q* at each time step.

During Q-Learning, experience tuples (otherwise known as records or memories) eᵢ:=(s, a, r, s′) of the agent's interaction with its environment are usually stored in a memory [symbol] . Each record is composed of an initial state s, the chosen action a, the received reward r an next state s′. During learning the agent samples records from past experiences and learns the optimal Q-Value function by minimizing the temporal difference error (TD-Error), defined as the difference between a target Q-Value (or target state-action value) computed from a record eᵢ and the current Q-Value for a particular state-action pair (s, a)∈eᵢ:

$${Q}^{*} ( s , a ) = {argmin}_{Q} {𝔼}_{{{e}_{i ∼ ℳ}}_{s , a ∈ {e}_{i}}} [ {Q}^{T} ( s , a \vert {e}_{i} ) - Q ( s , a ) ]$$

The target Q-Value (Q^{T}) is computed from an experience tuple eᵢ by combining the actual observed reward, and the maximum future expected reward.

$${Q}_{θ}^{T} ( s , a \vert {e}_{i} ) = \{ \begin{matrix}r & i f a is terminal \\ r + y {max}_{{a}^{′}} Q ( {s}_{i}^{′} , {a}^{′} ) & otherwise\end{matrix}$$

In practice, the Q-Values are updated iteratively from point samples until convergence or until a maximum number of steps is completed. At each iteration, the new Q-value is then defined as

Q(s,a)←(1−α)Q(s,a)+αQ^{T}(s,α|eᵢ)

with α the learning rate of the agent.

Notice that this method requires the value of Q for each state-action pair (s, a) to be stored. Hence, the classic Q-Learning algorithm and other tabular reinforcement learning methods often fall short in settings with large state-action spaces, which strongly constrain its potential use in healthcare. For example, in the specific implementations, the state space has |S|=3⁷⁷² possible configurations, corresponding to 597 elements of the set of observable medical evidence ε (symptom or risk factor).

The set ε may correspond to a subset of the clinical evidences each of which is in one of three states: unobserved, observed present, or observed absent. In specific embodiments, ε corresponds to the subset of clinical evidences used by the probabilistic graphical model (PGM) in production at Babylon Health.

Deep Q-Learning

Deep Reinforcement Learning refers to a series of new reinforcement learning (RL) algorithms that employ (Deep) Neural Networks (NNs) to approximate essential functions used by the agent. NNs amortize the cost of managing sizeable state-action space, both in terms of memory and computation time, and allow to learn complex non-linear functions of the state.

NNs are used in particular to learn the policy function directly or to learn a value function. Deep RL is better suited to handle the complex state-space associated with healthcare-related tasks. Those tasks often require reasoning over large state spaces of structured inputs composed of healthcare events, medical symptoms, physical signs, lab tests, or imagery results.

Deep Q-Learning is an approach that uses a NN to learn the Q-value of the state-action pairs Q_{θ} (s, a), with θ the parameters of the network. The core of the approach remains similar to classic Q-Learning but now uses stochastic gradient descent, rather than an explicit tabular update, to update θ following the gradient that minimizes the squared TD-error for each batch ξⱼ⊂ [symbol] :

$$ℒ ( {θ}_{j} ) = {𝔼}_{{{e}_{i} ∼ {ξ}_{j}}_{s , a ∈ {e}_{i}}} [ {( {Q}_{θ}^{T} ( s , a \vert {e}_{i} ) - {Q}_{{θ}_{j}} )}^{2} ]$$

$${θ}_{j + 1} = {θ}_{j} - {αΔ}_{{θ}_{j}} ℒ ( {θ}_{j} )$$

The embodiments described herein make use of a deep reinforcement learning approach to medical triage, where an artificial agent learns an optimized policy based on expert-crafted clinical vignettes.

An agent trained according to this process has been found to match human performance with an appropriate triage decision rate of 85% on previously unseen cases, and compared to a purely supervised method, has the advantage of learning a compressed policy by learning when to stop asking questions.

The methodology discussed herein does not train agents to ask specific questions. Instead, the approach can be used in conjunction with any question asking system, be it human, rule-based, or model-based.

Clinical Vignettes

The training and testing of the model relies upon a dataset of clinical vignettes, each describing a patient presentation Vᵢ:={vₖ|vₖ∈ε^{+/-}}. Each vₖ represents an instance of clinical evidence (a symptom or a risk factor, known to be either absent or present, or unknown). In certain embodiments |ε|J=1194, although any number of clinical evidences may be used to train the system. In a specific embodiment, each vignette Vᵢ is associated with a number of expert triage decisions:

Aᵢ={aⱼ^{m(a}^{ⱼ}⁾|αⱼ∈ [symbol] }

where [symbol] is the set of potential triage decisions (the set of triage classes) and m(aⱼ)∈ [symbol] is the multiplicity of a decision aⱼ in the multiset Aᵢ. These expert triage decisions are prelabelled classifications for each vignette (each vignette representing clinical evidence for a given person). These are determined in advance, by experts in the field.

In a specific embodiment, each vignette Vᵢ is associated with an average of 3.36 (standard deviation of 1.44) expert triage decisions, although other numbers of expert triage decisions may be used.

In a specific embodiment, the set of potential triage decisions [symbol] :={Red, Yellow, Green, Blue}. This four-color based system indicates how urgently a patient should be seen. It is similar to one used by the Manchester Triage Group (MTG) telephone triage, which simplifies to four categories the used 5-color triage system in the triage literature.

Red is associated with life-threatening situations, which requires immediate attention. Yellow indicates that the patient should be seen within the next couple of hours. Green that the patient should be seen but not urgently. Blue indicates that the patient should be given self-care advice and be directed towards a pharmacy, if necessary.

It should be noted that the exact classifications and names for each classification can vary, depending on the use-case. For instance, instead of colours, the triage level could be indicated by numbers (with numbers either ascending or descending relative to urgency).

Nevertheless, the general concept is that the user is assigned a triage level indicative of the urgency by which the patient should be processed or treated.

To ensure accuracy, the validity of each of the curated vignettes can be evaluated independently by (e.g. two) clinicians prior to training.

The triage decisions associated with each vignette can be determined from a panel of expert clinicians. For each vignette can be labelled with a triage decision, with the decisions being provided by separate clinicians, blinded to the true underlying disease of the presentation. The clinician's triage policy, which we aim to learn, can be left to their expertise and does not have to be constrained to a known triage system, such as the MTG.

The State-Action Space

In the task we are considering, at each time step the agent performs one of the available actions [symbol] ⁺:= [symbol] ∪ ask, where ask is the action of requesting more information. That is, the agent either asks for more information, or it makes one of the triage decisions.

For each vignette, the set of medical evidence Vᵢ is mapped to a full state vector representation Eᵢ∈S, with S:={−1,0,1}^{|ε|} being the state-space. The state-space S is a vector having each element taking discrete value of −1, 0 or 1. An element takes the value of −1 for known negative evidence (if the corresponding sign or risk factor is known to be absent, e.g., absence of fever), +1 for known positive evidence (e.g., headache present), and 0 for unobserved evidence. It is worth noting that expert-curated case cards are sparse, and many of the potential risk factors and symptoms are unobserved.

The Vignette Environment

At each new episode, the environment is configured with a new clinical vignette. The environment processes the evidence, and the triage decision on the vignette, and returns an initial state s₀ with only one piece of evidence revealed to the agent, i.e., s₀ is a vector of all zeroes of size |s₀|=|ε| except for one element which is either 1 or −1.

At each time step t, the environment receives an action aₜ from the agent. If the agent picks one of the triage actions, the episode ends, and the agent receives a final reward (discussed below). If the agent asks for more evidence, the environment uniformly samples one of the missing pieces of evidence and adds it to the state sₜ₊₁. During training, the agent is forced to make a triage decision if no more evidence is available on the vignette.

The Agent

In one embodiment, the agent architecture follows a Deep Q-Network (DQN) approach.

In a specific embodiment, the network is composed of four fully connected layers. The input layer takes the state vector sₜ∈{−1,0,1}^{|ε|}. The hidden layers are fully connected scaled exponential linear units (SeLU) layers with 1024 units. The output layer lₒᵤₜ∈ [symbol] uses a sigmoid activation function. Keeping lₒᵤₜ between 0 and 1 (restricting the output layer to a range between 0 and 1) allows for an easier process of reward shaping: by limiting the valid range for the rewards and treating them as probabilities of being the optimal action, rather than arbitrary scalar values.

Observations gathered by the agent are stored into a variant of the Prioritized Experience Replay Memory, in which experiences are prioritized by their temporal difference error. Observations are replayed in batches of 20 independent steps during optimization. After a burning period of 1000 steps, during which no learning occurs, the agent is then trained on a randomly sampled batch after each action.

To promote exploration during training, instead of using a classic ∈-greedy approach, a small amount of Gaussian noise ∈˜ [symbol] (0,σ(t)) added to Q(sₜ,ask) before the greedy policy picks the action with the highest Q-value:

$${a}_{t} = arg {max}_{a} ( {Q}_{θ} ( {s}_{t} , a ) + [ a = ask ] 𝒩 ( 0 , σ ( i ) ) )$$

where the operator [a=ask] is the Iverson bracket, which converts any logical preposition into a number that is 1 if the proposition is satisfied, and 0 otherwise. The noise standard deviation σ(t) a decayed an upper limit to a lower limit. In one embodiment, the noise standard deviation σ(t) is decayed from σ(t₀)=0.05 initially to σ(t_{>3000})=0.001.

The noise is added to the action ask and not to the triage actions [symbol] because the goal of exploration is to evaluate when to stop rather than to gather information about specific triage rewards. Here, the triage actions are terminal, and all receive a counterfactual reward, which is independent of the action picked at each time step.

The above noise function is simple to execute, thereby providing a computationally efficient process, whilst allowing effective exploration.

Counterfactual Reward

One key difference with other reinforcement learning settings is that the rewards are not delayed, and akin to a supervised approach, each action receives a reward, whether the agent performed that action or not.

At each time step, the reward received by the agent is then not a scalar, but a vector R∈ [symbol] , which represents the reward for each of the possible triage actions. The ask action does not receive a reward from the environment (as discussed below).

The reward informs all of the agent's actions, rather than only the single action it selected, as if it had performed all actions at the same time in separate counterfactual worlds.

Reward shaping is important for this task, and many reward schemes have been tested to fairly promote the success metrics of Appropriateness and Safety (discussed in more detail below). Trying to balance their relative importance into the reward proved to be less efficient than trying to match the distribution of expert's triage decisions.

Hence, for every vignette Vᵢ, each triage decision a∈ [symbol] is mapped to a reward equal to the normalized probability of that decision in the bag of expert decisions Aᵢ. Namely, denoting of r corresponding to the reward for action a as rₐ:

$${r}_{a} := r ( a , s \vert {A}_{i} ) = \frac{P ( a \vert {A}_{i} )}{{max}_{{a}^{′}} P ( {a}^{′} \vert {A}_{i} )} , ∀ s ∈ S$$

Moreover, since all triage actions are terminal, only the reward participates in the target Q-value for triage actions:

∀a∈ [symbol] ,Q^{T}(s,a|eᵢ)=rₐ

Consequently, to account for the counterfactual reward, the system uses a vector form of the temporal difference update where all actions participate in the error at each time step.

The reward for the action ask is treated differently. As described in the next section, it is defined dynamically based on the quality of the current triage decision, to encode the notion that the agent should be efficient, yet careful to gather sufficient information.

Dynamic Q-Learning

One key difference of the current methodology over the classic Q-Learning approach is the dynamic nature of Q_{θ}^{T} (s, ask|eᵢ), the large Q-value for the action ask, which depends on the current Q-values of the triage actions.

This dynamic dependency is especially useful given that the stopping and the triage part of the present Dynamic Q-Learning (DyQN) agent are being learnt at the same time, and the value of asking for more information might change as the agent becomes better at triage.

The ideal stopping criterion would stop the agent as soon as its highest Q-value corresponds to a correct triage decision, and do so reliably over all the vignettes. Assuming that the Q-values for the triage decisions are a good estimate of the probability of a particular triage, the DyQN approach is a heuristic which allows the agent to learn when best to stop asking questions given its current belief over the triage decisions. Two such heuristics are developed herein in the form of probabilistic queries.

The OR Query

The OR query is used by the DyQN: OR QUERY agent, as well as by the baseline agent PARTIALLY—OBSERVED: OR QUERY. In practice, during each optimization cycle and for each sampled memory eᵢ in the batch, the Q-Values for the starting state s and following state s′ are computed. Given the parameters 6 of the neural network, for state s, the maximum Q-value from the Q-values for the triage actions is referred to as:

$${Q}_{m} ( s ) = {max}_{a ∈ 𝒜} {Q}_{θ} ( s , a )$$

The target Q-value for asking is defined as:

Q^{T}(s,ask|eᵢ)=1−Qₘ(s)+Qₘ(S)Qₘ(s′)

For simplicity, we define Q̅ₘ(s)=1−Qₘ(s). Following this:

Q^{T}(s,ask|eᵢ)=Q̅ₘ(s)+Q̅ₘ(s)Qₘ(s′)

It can be seen that this definition can be loosely mapped to the classic target Q-value, if one considers r(ask, s)=Q̅ₘ(s) and γ=Qₘ(s).

To understand the origin of the above equation for the target Q-value, we must treat 0-values as probabilities and define the events T and T′ are defined as “the agent's choice is an appropriate triage” on the current state s and next state s′ respectively. Writing the event T̅ as the negation of T, the probability asking is defined as

Q^{T}(s,ask|eᵢ):=P(ask|s)=P(T̅∨T′|s,s′)

that is, the probability of the event “Either the triage decision is not appropriate in the current state, or it is appropriate in the next state”. The query can also be written as:

P(T∨T′|s,s′)=1−P(T∧T′|s,s′)

which shows that the OR query encodes a stopping criterion heuristic corresponding to the event: “The triage decision is appropriate on the current state, and not appropriate in the next state”.

If the Q-values for the triage actions are considered as probabilities Q(s,aᵢ)=P(T|s,a), then:

$$\begin{matrix}P ( T \vert s ) = {\sum }_{a ∈ 𝒜} π ( a \vert s ) P ( T \vert s , a ) \\ = {Q}_{m} ( s )\end{matrix}$$

This allows us to convert probabilities into Q-values. The probability of an appropriate triage (dependent on the Q-value for that triage) is linked to the probability ground of the triage within the ground truth values (the curated list of correct triages for the patient).

Assuming the Markov property and ensuing conditional independencies (T∥s′, T′|s) and (T′∥s|s′), the query can be rewritten as:

$$\begin{matrix}P ( \overline{T} ⩔ {T}^{′} \vert s , {s}^{′} ) = P ( \overline{T} \vert s ) + ( {T}^{′} \vert {s}^{′} ) - P ( \overline{T} ⩓ {T}^{′} \vert s , {s}^{′} ) \\ = P ( \overline{T} \vert s ) + ( {T}^{′} \vert {s}^{′} ) - P ( \overline{T} \vert s ) P ( {T}^{′} \vert {s}^{′} ) \\ = {\overline{Q}}_{m} ( s ) + {Q}_{m} ( {s}^{′} ) - {Q}_{m} ( {s}^{′} ) ( 1 - {Q}_{m} ( s ) ) \\ = {\overline{Q}}_{m} ( s ) + {Q}_{m} ( s ) {Q}_{m} ( {s}^{′} )\end{matrix}$$

The AND Query

The AND query is used by the DyQN: AND QUERY and the PARTIALLY—OBSERVED: AND QUERY baselines (discussed later in the Results section). For this query, the Q-value target for the ask action is defined as:

Q_{θ}^{T}(s,ask|eᵢ)=Q̅ₘ(s)(Qₘ(s′)+Q̅ₘ(s)Qₘ(s′)Q_{θ}(ask|s′))

Contrary to the OR query, which can be viewed as a particular parametrisation of the reward and of the classic Q-Learning target, the AND query has a form which is not immediately comparable.

In this case, the Q-target is obtained by considering the sequence of the event T until the end of the interaction. That is, we consider the events Tⱼ, Tⱼ₊₁, . . . , Tₖ, for the states sⱼ up to sₖ, with k+j the maximum number of questions. We then consider the probability Pⱼ of the event “The current triage decision is incorrect, and the next is correct, or both the current and next triage decision are incorrect, but the following triage decision is correct, or . . . ” and so on. We can rewrite Pⱼ as:

$$\begin{matrix}{P}_{j} = P ( {{⩔}_{m = j + 1}}^{k} [ {T}_{m} {{⩓}_{n = j}}^{m - 1} {\overline{T}}_{n} ] \vert {s}_{j} , {s}_{j + 1} ) \\ = P ( {\overline{T}}_{j} ⩓ {T}_{j + 1} \vert {s}_{j} , {s}_{j + 1} ) + \\ P ( {\overline{T}}_{j} \vert {s}_{j} ) P ( {{⩔}_{m = j + 2}}^{k} [ {T}_{m} {{⩓}_{n = j + 1}}^{m - 1} {\overline{T}}_{n} ] \vert {s}_{j} , {s}_{j + 1} ) - \\ \underbrace{P ( {{⩓}_{m = j + 1}}^{k} [ {T}_{m} {{⩓}_{n = j}}^{m - 1} {\overline{T}}_{n} ] \vert {s}_{j} , {s}_{j + 1} )}_{= 0} \\ = P ( {\overline{T}}_{j} \vert {s}_{j} ) ( P ( {T}_{j + 1} \vert {s}_{j + 1} ) + P ( {\overline{T}}_{j + 1} \vert {s}_{j + 1} ) {P}_{j + 1} ) \\ = {\overline{Q}}_{m} ( {s}_{j} ) ( {Q}_{m} ( {s}_{j + 1} ) + {\overline{Q}}_{m} ( {s}_{j + 1} ) {Q}_{θ} ( ask \vert {s}_{j + 1} ) )\end{matrix}$$

In practice, for both AND and OR queries, better results can be obtained by using the known appropriate triages A^{e}^{ᵢ} in each of the sampled memories et and defining

$${Q}_{m} ( s ) := {Q}_{m} ( s \vert {e}_{i} ) = {max}_{a ∈ {A}^{{e}_{i}}} {Q}_{θ} ( s , a )$$

that is, the maximum Q-value associated with an appropriate triage.

Whilst the OR query has provided the best results so far, the AND query provides the best results regarding the stopping criterion out of the other queries tests. Whilst theoretically, the AND query should be more accurate, the OR query has been found to provide more appropriate and safe triages. This is likely due to the fact that the AND query assumes a perfect model for assessing appropriate triages (e.g. a perfect neural network). In this case, more evidence would always improve performance. In contrast, it has been found that obtaining more evidence can sometimes negatively affect performance (produce a less safe or less appropriate triage result). The OR query takes this into account, learning to stop at the most appropriate time.

Memory

The agent's memory is inspired from Prioritized Experience Replay Memory (PER) but does not rely on importance weighting. Instead, each memory tuple eᵢ:=(s, a, r, s′) is associated with a priority:

$${v}_{i} = \vert \frac{1}{\vert 𝒜 \vert} {\sum }_{a ∈ 𝒜} {Q}_{θ θ}^{T} ( s , a \vert {e}_{i} ) - {Q}_{θ} ( s , a ) \vert$$

which relies on the vector form of the counterfactual reward r and is equal to the absolute value of the mean TD-Error over every action. The experience tuple eᵢ is stored along with its priority vᵢ, which determines in which of the priority buckets the memory should be stored. The priority buckets have different sampling probabilities. In one embodiment, the sampling probabilities range from 0.01 for the lowest probability bucket to 0.8 for the highest.

Before each optimisation step, a number (e.g. 200) of experience tuples are sampled from the priority buckets, and every time a tuple is sampled, its priority decays with a factor λ (in one embodiment, λ=0.999) which slowly displaces it into priority buckets with lower sampling probability. This approach contrasts with the priority update of PER, which sets the priority equal to the new TD-Error computed during the optimisation cycle. This approach yields better empirical results than using the classic PER priority update and importance weighting.

### Embodiments

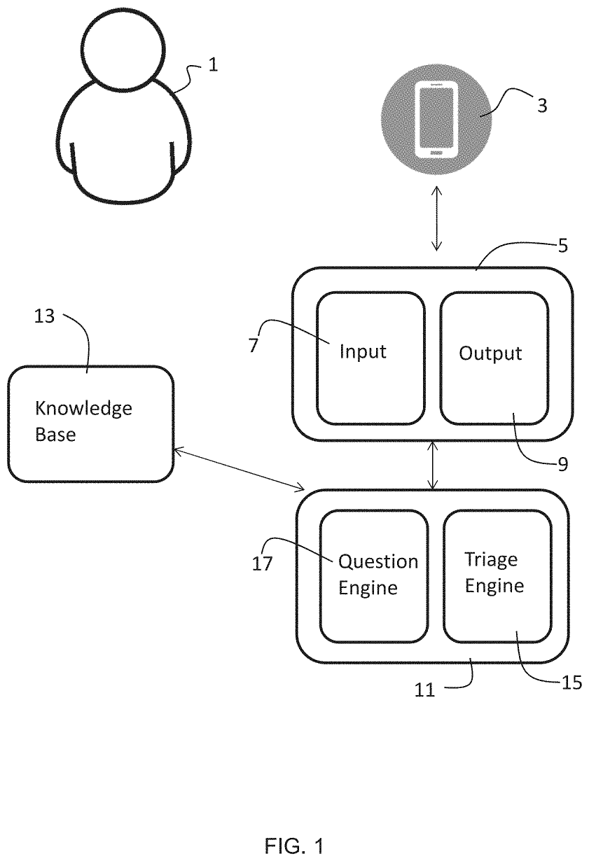

FIG. 1 shows a schematic of a triage system according to an embodiment. In one embodiment, a user 1 (a patient) communicates with the system via a mobile phone 3. However, any device could be used that is capable of communicating information over a computer network, for example, a laptop, tablet computer, information point, fixed computer, voice assistant, etc.

The mobile phone 3 communicates with interface 5. Interface 5 has two primary functions; the first function 7 is to take the words input by the user and turn them into a form that can be understood by the triage system 11. These words may be text that is input (e.g. typed) into the mobile phone. Alternatively, these words may be spoken (uttered) by the user and recorded by the phone, for instance, via a microphone. The second function 9 is to take the output of the triage system 11 and to send this back to the users mobile phone 3.

In the present embodiments, Natural Language Processing (NLP) is used in the interface 5. NLP is one of the tools used to interpret, understand, and then use every day human language and language patterns. It breaks speech or text down into shorter components and interprets these more manageable blocks to understand what each individual component means and how it contributes to the overall meaning.

The triage system 11 comprises a triage engine 15 and a question engine 17. The question engine 17 is configured to generate questions to obtain information regarding the user (i.e. evidence). In this way, the system obtains evidence of potential medical conditions of the user (e.g. in the form of positive or negative indications or certain symptoms or risk factors).

The triage engine 15 is configured to assess the evidence obtained so far and either 1) determine a triage classification for the user or 2) prompt the question engine 17 to issue another question to obtain further evidence. Accordingly, the triage engine 15 assesses whether sufficient evidence has been obtained to provide a reliable triage decision and, if not, requests further evidence.

The question engine 17 may be configured to determine the most effective question to ask in order to improve the accuracy of the triage decision and/or a subsequent diagnosis of the user. To achieve this, the question engine 17 may be configured to refer to a knowledge base 13.

The question engine 17 may be implemented through a probabilistic graphical model that stores various potential symptoms, medical conditions and risk factors. The question engine may applies logical rules to the knowledge base 13 and probabilistic graphical model to deduce new information (infer information from the input information, the knowledge base 13 and the probabilistic graphical model). The question engine is configured to generate questions for the user to answer in order to obtain information to answer an overall question (e.g. “what is the triage level”). Each question is selected in order to reduce the overall uncertainty within the system.

In the present case, the triage engine 15 utilises the question engine 17 to determine a triage level for the user (e.g. urgent, not urgent, etc.). The question engine 17 selects a question by choosing the question that would most increase the value of information (i.e. that would most decrease uncertainty in the triage decision or in a subsequent diagnosis). The user's answer is then passed back to the triage engine 15 that uses this new information to either determine a triage level for the user or prompt a further question.

In an embodiment, the knowledge base 13 is a large structured set of data defining a medical knowledge base. A knowledge base is a set of facts and rules that the system has access to for determining a triage level. The knowledge base 13 describes an ontology, which in this case relates to the medical field. It captures human knowledge on modem medicine encoded for machines. This is used to allow the above components to speak to each other. The knowledge base 13 keeps track of the meaning behind medical terminology across different medical systems and different languages. In particular, the knowledge base 13 includes data patterns describing a plurality of semantic triples, each including a medical related subject, a medical related object, and a relation linking the subject and the object.

An example use of the knowledge base 13 would be in automatic diagnostics, where the user 1, via mobile device 3, inputs symptoms they are currently experiencing, and the interface engine 11 identifies possible causes of the symptoms using the semantic triples from the knowledge base 13. Following triage, the system may also proceed to diagnose the patient, for instance, by requesting further information before determining a likely condition, although diagnosis is not an essential feature of the present disclosure.

The present embodiments are directed towards the triage engine 15 that aims to determine an appropriate and safe triage level for the user and that prompts the question engine 17 to issue questions to obtain more evidence, where needed. As discussed above, this utilises an artificial agent trained using reinforcement learning.

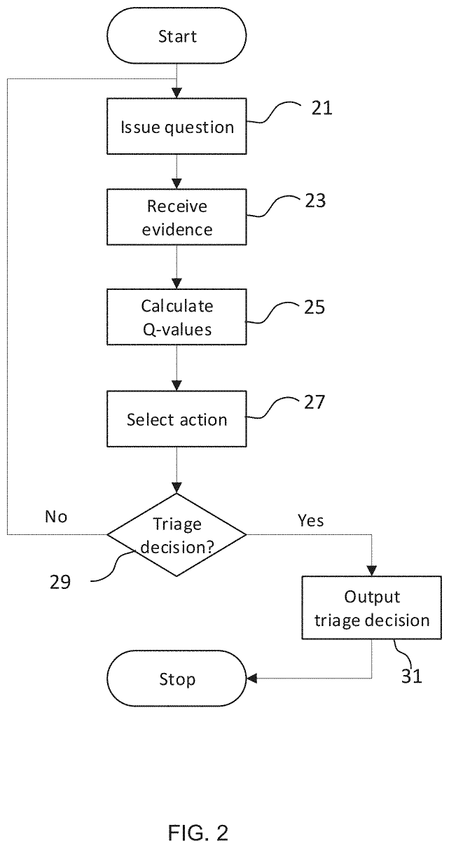

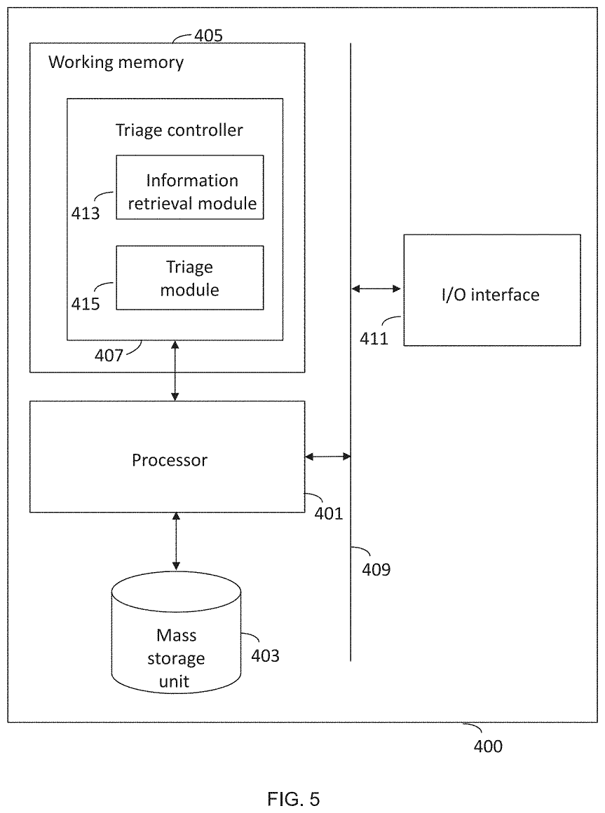

FIG. 2 shows a method of determining a triage level using an artificial agent trained according to an embodiment. The method may be implemented by the system of FIG. 1 (e.g. by the triage engine 15), or may be implemented by any other form of computing system, for instance, that of FIG. 5. For completeness, the following shall be described with regard to the triage engine 15; however, it will be appreciated that this method is not limited to this specific implementation.

The method starts by the system issuing a question 21 to the patient. In the embodiment of FIG. 1, the question engine 17 generates a question and sends it to the user's device 3. The user enters an answer (e.g. through a multiple-choice selection or through a natural language input). This answer is passed back to the triage engine 15 via the interface 7 which may, in turn, convert the input into a format appropriate for the triage engine, for instance, by mapping the input to a vector of evidence Vᵢ, described above.

The evidence is a vector where each entry relates to a specific observation of a given attribute of the patient (an individual piece of evidence).

When the triage engine 15 receives the evidence, it then calculates the Q-values for the potential actions 25 (the potential triage actions and the asking action). This is determined by inputting the state (the current evidence vector including the observed attributes of the patient) into a neural network trained to predict a Q-value for each of the action based on the parameters of the network.

The action is then selected 27. As discussed in section “The Agent”, the selected action is that which provides the highest Q-value. During training, noise may be added to the Q-value for the asking action (the information request action) to promote exploration.

The system then determined whether the selected action was a triage action 29. If not, then action was an asking action (an information retrieval action) and the method loops back to step 21 to issue a subsequent request for information in the form of a follow-up question. In the case of the system of FIG. 1, the triage engine 15 issues a commend to the question engine 17 to generate and send a further question.

If a triage action is selected 29, then the triage class of the user is output 31. This might be output to the user to inform them of their level of triage and provide appropriate advice, for instance, to direct them to a doctor or hospital where an urgent triage level is determined, or to direct them to self-medicate, for instance, through visiting a pharmacy for a low triage level. In addition, the user may be placed in a queue for treatment or to see a medical professional (e.g. a nurse or doctor) based on the triage level.

In addition to determining whether the selected action is a triage action is step 29, the system may also determine if a maximum number of questions has been issued. If so, then the user may be informed of this and the method may end. In one embodiment, if the maximum number of questions has been reached, then the triage level with the highest confidence may be selected, although alternative embodiments may issue (either in conjunction with this triage level or alternatively to providing this triage level) and indication that the maximum number of questions has been reached without a sufficiently accurate triage level being determined.

The above methodology allows the system to make appropriate triage decisions when sufficient evidence has been obtained and to ask additional questions where more evidence is needed. This relies on a trained neural network that is configured to predict Q-values (state-action values) for each potential action (triage actions or information request actions).

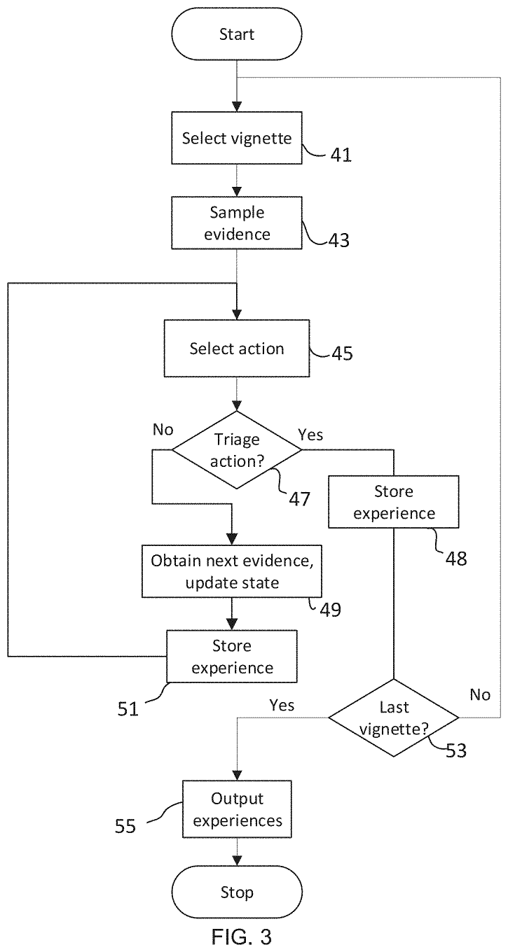

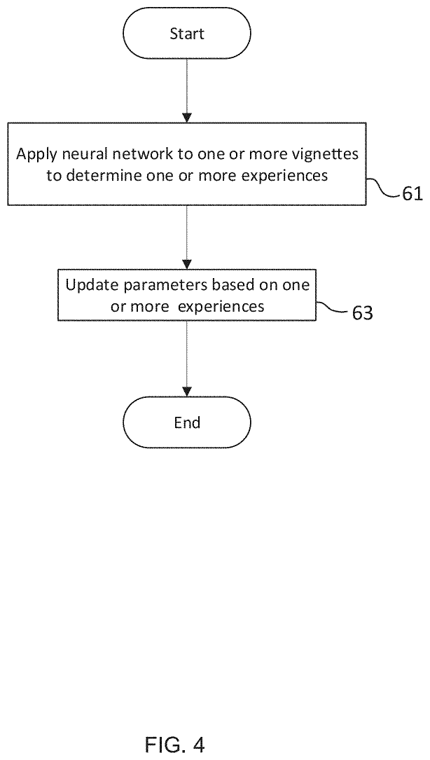

FIG. 3 shows a method of determining experiences for training a triage system according to an embodiment. This method incorporates a number of the steps of FIG. 2 but has been adapted for an offline implementation that does not interact with patients and instead learns to take actions based on pre-prepared vignettes of evidence and predetermined triage levels. Once the experiences have been obtained, the neural network can be trained, as shown in FIG. 4.

The method starts with the selection of a vignette 41 representing a set of evidence of observed attributes of a specific patient. The vignette contains the full set of evidence known about the patient. A subset of evidence is sampled from the vignette 43. This forms a state for the environment. In a specific embodiment, one piece of evidence is sampled at a time. An action is then selected 45. This is selected by choosing the action that has the highest Q-value. That is, the state (the sampled evidence) is input into the neural network for calculating Q-values and an action is selected in a similar manner to that discussed with regard to FIG. 2. Noise may be added to the Q-value for the ask action to promote exploration.

The method determines whether the action is a triage action 47. If so, the analysis of the vignette ends, the experience of this action is stored 48 and the method moves to step 53 (discussed below) to determine whether the last vignette has been considered or whether further vignettes are to be analyzed. The experience for a triage action includes at least the state, the corresponding action and a reward for one or more of the triage actions. As the triage action is terminal, and as the Q-values for triage actions are equal to the reward and are not dependent on the next state, there is no need to update the state by adding new evidence, but instead the next state may be deemed to be the same as the current state. Accordingly, the next state may be stored in the experience but may be set to be the same as the state for the experience.

If the action is an ask action then the ask action is implemented by requesting additional evidence and the state is updated 49. In this case, the additional evidence is obtained (sampled) from the vignette. If no additional evidence is available, or if a maximum number of ask actions have been performed, then a triage decision is forced and the method moves to step 53. Forcing a triage action includes the selection of the triage with the highest Q-value, regardless of the Q-value for the ask action. A triage decision is forced on the agent by the training environment returning an indication that no further evidence is available and a command for a triage decision to be made.

The update to the state includes the addition of a new set of evidence from the vignette to the state in the event that an information request action is selected.

The experience of this action is then stored 51. Each experience for an ask action includes at least the state, the corresponding action, the updated state and a reward for one or more of the triage actions

For each experience (whether for a triage or ask action), a counterfactual reward may be stored relating to all potential actions. In this embodiment, a reward is stored for all triage actions (regardless of which action is selected), to provide a counterfactual reward across all triage actions. In another embodiment, the reward is only stored for a predefined selection of triage actions that are predefined as correct (e.g. safe or appropriate). This provides improvements in the efficiency and accuracy of the method.

It should be noted that, in many cases, the rewards will not need to be calculated at each step, as they will not change. Instead, the rewards are based on the probability of the respective triage level being correct based on a predetermined set of triage levels for the patient (see “Counterfactual Reward” section). Accordingly, whilst step 51 states that a reward is stored, there may be no need to repeatedly store the same reward, and instead, each experience may be stored as a state, an action and a next state, with reference to a single stored instance for each relevant reward for the patient. The rewards may be calculated as part of the method (based on the predetermined triage levels for the patient) or may calculated by an external system and provided as an input to the method.

Following the storage of the experience tuple 51 the parameters of the neural network may be updated based on the stored experiences. Having said this, in the present embodiment, the update occurs after a number of experiences have been stored (see FIG. 4).

Once the experience has been stored, the method loops back to step 45 to select a new action.

Once a triage action has been determined, then the method determines whether an end criterion has been reached for the analysis (e.g. a maximum number of vignettes have been processed). If not, then the method loops back to step 41 to select a new vignette for a new patient. If the end of analysis has been reached, then the experiences are output 55 (e.g. stored or sent to an external system for future use).

The experiences can be used to update the parameters of the neural network to train the neural network to determined more accurate actions.

FIG. 4 shows a method of training a triage system according to an embodiment. The method starts with the application of the neural network to one or more vignettes to determine one or more experiences 63. This may be through the method of FIG. 3. The parameters are then updated 63 based on one or more of the obtained experiences. After the update, the method may loop back to step 61 to determine one or more further experiences based on the updated parameters.

As discussed herein, the update may occur after a predetermined number of initialization steps, where the agent runs without any updates. After this predetermined number, the stored experiences may then be sampled and the update performed for each iteration of the agent (or the update may be performed without performing any further actions by the agent). The sampling may occur based on a relative priority of each experience, calculated based on an error (e.g. a time difference error) for the experience (which, in this case, is calculated and stored with the experience).

Whilst a number of methods for updating the network parameters are available, the present embodiment makes use of temporal difference learning. In this embodiment, a target Q-value (Q^{T}) is calculated for each potential action. As discussed in the “Counterfactual Reward” section, the update in this embodiment makes use of a target Q-value for every potential action, regardless of whether this action was selected.

The Q-values for each triage action are equal to the reward for taking the action. This is proportional to the probability of that triage decision within the predetermined triage decisions for the vignette:

$${Q}^{T} ( S , a \vert {e}_{i} ) = {r}_{a} := r ( a , s \vert {A}_{i} ) = \frac{P ( a \vert {A}_{i} )}{{max}_{{a}^{′}} P ( {a}^{′} \vert {A}_{i} )} , ∀ s ∈ S$$

That is, this is equal to the normalized probability (normalized relative to the maximum probability across the potential actions).

In the specific embodiment where only a selection of triage actions (e.g. predefined appropriate, correct or safe triage actions for the patient) have rewards stored, then the Q-values for triage actions are only calculated for each reward stored (each triage action of the selection of triage actions).

The target Q-value for the information request action (the ask action) is based on the target state-action values for the triage actions. As the target Q-values for the triage actions are based on a probability (i.e. a probability that the triage action is correct), the target Q-value for the information request action can therefore be a probability that more information is needed based on the current Q-values for triage. This can be considered a probability that the agent will make an incorrect triage decision for the current state (i.e. that the maximum probability/maximum Q-value triage action is inappropriate or otherwise incorrect). Furthermore, this may also include a probability that the agent will make a correct triage action for a future state (e.g. that the maximum probability/maximum Q-value triage action is appropriate or otherwise correct for the next state).

This can be calculated based on the maximum of the calculated Q-values for the relevant triage actions (the probability of the maximum probability triage action that would be selected, if a triage action were to be taken):

$${Q}_{m} ( s ) = {max}_{a ∈ 𝒜} {Q}_{θ} ( s , a )$$

The Q-value for the asking action is based on the maximum triage Q-value for both the current state and next state in the given experience.

Note that, whilst the above equation specifies the maximum of all of the triage actions, in the embodiment where only a subset of triage actions are considered (a subset of predetermined appropriate triages for the patient), this maximum may be the maximum from this subset.

In one embodiment, the Q-value for the information request action is determined based on the OR query discussed in the section “The OR Query”. Specifically, the target Q-value for asking is defined as:

Q^{T}(s,ask|eᵢ)=Q̅ₘ(s)+Q̅ₘ(s)Qₘ(s′)
where

Q̅ₘ(s)=1−Qₘ(s)

More specifically, this is a probability that either the agent will make an incorrect triage decision for the current state in the experience or that the agent will select a correct triage decision for the next state. The agent selects a triage decision if it has the maximum Q-value. Accordingly, this is a probability that either the maximum Q-value for the state relates to a triage action that is inappropriate given the state or that a maximum Q-value for the next state relates to a triage action that is appropriate given the next state

In one embodiment, the Q-value for the information request action is determined based on the AND query discussed in the section ‘The AND Query’. Specifically, the target Q-value for asking is defined as:

Q_{θ}^{T}(s,ask|eᵢ)=Q̅ₘ(s)(Qₘ(s′)+Q̅ₘ(s)Qₘ(s′)Q_{θ}(ask|s′))

More specifically, this is a probability that that the probability that a maximum state-action value (maximum Q-value) for a subsequent state relates to a triage action that would be correct given the subsequent state and that the maximum state-action value for each state before the subsequent state relates to a triage action that is incorrect given the corresponding state.

Algorithm 1 Training cycle of the Dynamic Q-Network (DyQN)

```
Require: DyQN’s Q_{θ}(s, a) and Q [symbol]  (s,a | e) functions, environment’s step(a), memory’s store
    (s, a, r, s′, v) and sample(size), noise variance σ( [symbol]  ).
Input: dataset [symbol]     of clinical vignettes
  1: Initialization θ ← θ₀
  2: for i ← to N do [symbol]    until the maximum number of games is reached. [symbol]
  3:  Vᵢ ~ [symbol]
  4: [symbol]    = step(Ø)
  5:  stop = False
  6:  for k ← 0 to K do [symbol]     until maximum question is reached. [symbol]
  7:  if stop then [symbol]    the environment forced a triage action. [symbol]
  8:  A = [symbol]
  9:  else
10:  A = [symbol] ⁺ = [symbol]   ∪ ask
11:  end if
12:   $\text{?} = {arg max}_{\text{?}} Q ( , a ) [ a = ask ] 𝒩 ( 0 , σ ( i ) ) \{ \text{noise is added to the}$
  Q-value for ask before greedy selection. [symbol]
13: [symbol]   , rₖ, stop = [symbol] (aₖ)
14: [symbol]    = (sₖ, aₖ, [symbol] , [symbol] )
15:  $= \vert ( , a \vert ) - Q ( , a ) \vert ( \text{compute memory priority.} )$
16: [symbol]
17:  if i ≥ L then [symbol]    after the burn-in period perform one optimization cycle at each step. [symbol]
18:  ξ = sample(N) [symbol]    Sample a batch of size N from memory. [symbol]
19:   $ℒ ( θ ) = {( ( s , a \vert {e}_{i} ) - Q ( s , a ) )}^{2}$
20:  θ ← θ − [symbol] [symbol]     (θ)
21:  end if
22:  if [symbol]    ≠ ask then [symbol]    sample new vignette when a triage decision is made. [symbol]
23:  break
24:  end if
25:  end for
26: end for [symbol]
indicates data missing or illegible when filed
```

Regardless of how the Q-value for the information request action is determined, the update is performed based on the error (e.g. the difference) between each target Q-value and its respective Q-value:

$$ℒ ( {θ}_{j} ) = {𝔼}_{{{e}_{i} ∼ {ξ}_{j}}_{s , a ∈ {e}_{i}}} [ {( {Q}^{T} ( s , a \vert {e}_{i} ) - {Q}_{{θ}_{i}} )}^{2} ]$$

$${θ}_{j + 1} = {θ}_{j} - {αΔ}_{{θ}_{i}} ℒ ( {θ}_{j} )$$

Once the parameters have been updated,

The above method is shown in more detail in Algorithm 1.

While the reader will appreciate that the above embodiments are applicable to any computing system, a typical computing system is illustrated in FIG. 5, which provides means capable of putting an embodiment, as described herein, into effect. As illustrated, the computing system 400 comprises a processor 401 coupled to a mass storage unit 403 and accessing a working memory 405. As illustrated, a triage controller 407 is represented as a software product stored in working memory 405. However, it will be appreciated that elements of the LM controller 407 may, for convenience, be stored in the mass storage unit 403.

Usual procedures for the loading of software into memory and the storage of data in the mass storage unit 403 apply. The processor 401 also accesses, via bus 409, an input/output interface 411 that is configured to receive data from and output data to an external system (e.g., an external network or a user input or output device). The input/output interface 411 may be a single component or may be divided into a separate input interface and a separate output interface.

The triage controller 407 includes an information retrieval module 413 and a triage module 415. The information retrieval module 413 is configured to obtain evidence about a patient. This might be through requesting or generating questions for the patient, or might be through accessing information within a set of stored evidence (e.g. for training). The triage module 415 is configured to determine a triage level of the patient over one or more rounds of information retrieval. The triage module 415 includes a neural network that is configured to predict the value of certain actions in order to assist with this triage. The neural network may be pretrained for this function and/or may include functions for updating the parameters of the neural network based on performance.

The triage controller software 407 can be embedded in original equipment or can be provided, as a whole or in part, after manufacture. For instance, the triage controller software 407 can be introduced, as a whole, as a computer program product, which may be in the form of a download, or be introduced via a computer program storage medium, such as an optical disk. Alternatively, modifications to an existing controller can be made by an update, or plug-in, to provide features of the above described embodiment.

The mass storage unit 403 may store the parameters of the neural network for access by the triage module 415. The mass storage unit 403 may also store evidence regarding one or more patients and, for each patient, a set of predetermined triage levels. This information may be used by the triage module to train the neural network based on its performance relative to the predetermined triage levels, as described herein.

The computing system 400 may be an end-user system that receives inputs from a user (e.g., via a keyboard or microphone) and determines outputs to the inputs based on the language model. Alternatively, the system may be a server that receives inputs over a network and determines corresponding outputs, which are then conveyed back to the user device.

The embodiments described herein provide methodology for training a computing system to perform triage on patients, assigning a triage level to each patient based on evidence obtained about the patient. The computing system implements an artificial agent which selects from actions including asking for more information and assigning of one of a set of triage levels. The agent is trained to learn when to continue asking for more information and when to decide on a specific triage level.

By training the system according to the embodiments described herein, the triage system can be trained to make triage decisions that rival and sometimes improve upon triage decisions made by human experts.

Discussion of Results

The present application presents a deep reinforcement learning approach (a variant of Deep Q-Learning) for triaging patients using curated clinical vignettes. The vignettes may be created by medical doctors to represent real-life cases.

The below discussion relates to the performance of a specific embodiment. In this embodiment, a dataset consisting of 1374 clinical vignettes was used, with each vignette being associated with 3.8 expert triage decisions given by medical doctors relying solely on medical history. This approach yields safe triage decisions in 94% of cases and matches expert decisions in 85% of cases. Furthermore, the trained agent learns when to stop asking questions leading to optimized decision policies requiring less evidence than supervised approaches, and adapts to the novelty of a situation by asking for more information, when required.

Overall, this deep reinforcement learning approach can learn strong medical triage policies directly from clinicians' decisions, without requiring expert knowledge engineering. This approach is scalable, inexpensive, and can be deployed in healthcare settings or geographical regions with distinct triage specifications, or where trained experts are scarce, to improve decision making in the early stage of care.

Metrics

The quality of the above embodiment has been evaluated on a test set composed of previously unseen vignettes, using three target metrics: appropriateness, safety, and the average number of questions asked. During training, those metrics were evaluated over a sliding window of 20 vignettes, and during testing, they were evaluated over the whole test set.

Given a bag (a set) of triage decisions Aᵢ, a triage a is defined as appropriate if it lies at or between the most urgent U(Aᵢ) and least urgent u(Aᵢ) triage decisions for each vignette. For instance, if a vignette has two ground truth triage decisions {Yellow, Blue} from two different doctors, the appropriate triage decisions are {Yellow, Green, Blue}.

A triage decision is considered safe if it lies at or above u(Aᵢ), the least urgent triage decision in Aᵢ. For instance, in the above example of a vignette have two ground truth triage decisions {Yellow, Blue}, the safe triage decisions are {Red, Yellow, Green, Blue}. Correspondingly, safety can be defined as the ratio of the agent's triage decisions that were safe over a set of vignettes.

The RL agent is trained to decide when best to stop and make a triage decision. Accordingly, the average number of questions can be used to assess the performance of the agent. The average number of questions is taken over a set of vignettes. In the present analysis, it varies between 0 and 23, an arbitrary limit at which point the agent is forced to make a triage decision.

Baselines

This reinforcement learning approach has been compared to a series of baselines—fully supervised approaches using the same train (N=1248) and test (N=126) split of the dataset [symbol] . The supervised models were voting ensembles of classifiers calibrated using isotonic regression.

A FULLY—OBSERVED model was tested. The FULLY—OBSERVED model was trained using the vignettes with their complete set of evidence Vᵢ. It represents the less optimized version of the triage policy, which can only deal with full presentations.

In addition to the two DyQn agents (OR query and AND query) defined above, two other agents, referred to as PARTIALLY—OBSERVED agents, were tested. The learning agents refers to the two DyQN agents and the two PARTIALLY—OBSERVED agents, because those four agents learn to stop during the RL training. Having said this, the triage actions of the PARTIALLY—OBSERVED agents were pretrained in a fully supervised way on a greatly expanded dataset of clinical vignettes [symbol] _{pow} constructed from the original set of vignettes [symbol] . Given a vignette Vᵢ∈ [symbol] , a new vignette was generated for each element of the powerset of the evidence set [symbol] (Vᵢ) with a cap at 2¹⁰.

If the vignette has more than ten pieces of evidence, the k=|Vᵢ|−10 remaining evidences generate k vignettes for each of the element of the powerset, by growing the rest of the evidence linearly and combining it with each element v∈ [symbol] (Vᵢ) For instance, if |Vᵢ|=12, two pieces of evidence {vₘ, vₙ}∈Vᵢ are sampled, and for each v∈ [symbol] (Vᵢ\{vₘ, vₙ}) one vignette will be created with evidence set v, another with v∪{ₘ}, and one with v∪{vₘ, vₙ}.

Using the described process, the system generated max(1, |Vᵢ|−10)×2^{min(|V}^{ᵢ}^{|,10)} new vignettes from each vignette Vᵢ belonging to the original dataset [symbol] . Critically, for each created vignette, the correct triage decisions are the same as the generating vignette.

After having trained a classifier on this extended dataset, the RL agent uses the class probabilities returned by the classifier as Q-values for the triage actions. In other words, only the ask action is trained during the RL phase. Hence, the PARTIALLY—OBSERVED agent does not improve on its ability to triage given a fixed set of evidence but uses the RL process to train a stopping criterion.

Two sub-types of PARTIALLY—OBSERVED agents are presented herein, the PARTIALLY—OBSERVED: OR QUERY, which uses the Q-value target defined above with respect to the OR query, and the PARTIALLY—OBSERVED: OR QUERY which uses the AND query defined above.

The embodiments were also compared to a RANDOM policy, which picks random actions, and ALWAYS—GREEN policy, which always picks the triage action Green, which had the highest prior probability in the dataset (0.48).

The human performance on the triage task with full evidence was estimated using a proxy metric called the sample mean inter-expert agreement for appropriateness Hₐ and safety Hₛ. For each vignette Vᵢ and each associated bag of expert decisions Aᵢ, this metric is the sample mean of the ratio of the experts' decisions a∈ [symbol] ᵢ, which were appropriate, or safe, given the decisions Aᵢ\a from the other experts. Here a represents an element of multiplicity 1 in the multiset, that is only one expert decision.

Human appropriateness and human safety are defined as:

$${H}_{a} = {𝔼}_{{V}_{i} ∼ 𝒟} [ \frac{1}{\vert {A}_{i} \vert} {\sum }_{a ∈ {A}_{i}} [ u ( {A}_{i} \text{\textbackslash} a ) \leq a \leq U ( {A}_{i} \text{\textbackslash} a ) ] ]$$

$${H}_{s} = {𝔼}_{{V}_{i} ∼ 𝒟} [ \frac{1}{\vert {A}_{i} \vert} {\sum }_{a ∈ {A}_{i}} [ u ( {A}_{i} \text{\textbackslash} a ) \leq a ] ]$$

Table 1 summaries the results of the testing.

| | Appropriateness | | Safety | | Avg. Questions | | N |
|---|---|---|---|---|---|---|---|
| DYQN: OR QUERY | .85 | (.023) | .93 | (.015) | 13.34 | (.875) | 10 |
| DYQN: AND QUERY | .76 | (.014) | .86 | (.012) | 1.40 | (.331) | 10 |
| PARTIALLY-OBSERVED: OR QUERY | .79 | (.000) | .88 | (.000) | 23 | (.000) | 10 |
| PARTIALLY-OBSERVED: AND QUERY | .79 | (.006) | .86 | (.005) | 10.35 | (.467) | 10 |
| RANDOM | .39 | (.027) | .74 | (.026) | .25 | (.025) | 10 |
| ALWAYS-GREEN | .71 | (.000) | .75 | (.000) | 0 | (.000) | 10 |

| | | | |
|---|---|---|---|
| HUMAN | .84 | .93 | full |
| FULLY-OBSERVED | .86 | .94 | full |

As can be seen, the proposed embodiments that utilize the OR query (DyQN: OR QUERY) provide comparable performance (in terms of appropriateness and safety) to human triage decisions based on the full set of evidence, whilst also learning when to stop asking questions, thereby reducing the amount of information that needs to be obtained.

The DyQN agent using the OR query performs better than the other agents in term of appropriateness (M=0.85, CI95=0.023, min=0.81, max=0.90) and safety (M=0.93, CI95=0.015, min=0.90, max=0.97). While it relies on less clinical evidence, asking on average 13.3 questions (CI95=0.015,min=10.8,max=15.1), it is on a par with human performance (0.84 appropriateness) as well as the fully-observed baseline (0.86 appropriateness), both of which use all the evidence on the vignette to come to a decision.

Accordingly, by learning when best to stop asking questions give a patient presentation, the DyQN is able to produce an optimised policy which reaches the same performance as supervised methods while requiring less evidence. It improves upon clinician policies by combining information from several experts for each of the clinical presentations. Moreover, while the result on the test set is on a par with human performance, the performance of the fully supervised approach on the training set (M=95 appropriateness) indicates that the task has a low Bayes Error rate, and given enough data we would expect DyQN to exceed human performance.

One of the reasons to use the Dynamic Q-Learning over classic Q-Learning is to ensure that the Q-values correspond to a valid probability distribution. Using the classic DQN would produce unbounded Q-values for the asking action, because asking is not terminal, whereas the Q-values for the triage action would be bounded. In classic Q-Learning, only a careful process of reward shaping for the ask action could account for this effect.

While the problem of optimal stopping has been studied in settings where actions are associated with a cost, the other immediate advantage of DyQN is that it is able to treat the stopping heuristic as an inference task over the quality of the agent's triage decisions. Interpreting the triage actions' Q-values as probabilities allows us to rewrite the Q-value update as the solution to the inference query, which leads to the agent getting increasingly better at it through interaction, and adapting dynamically as triage decisions improve during training.

This approach is well tailored for information gathering tasks, where an agent must make inference on a latent variable (here the triage) given the information it has gathered so far. Accordingly, whilst the above embodiments are described with respect to the specific task of classifying patient evidence to determine a triage level, the methodology is not limited in this regard, and can be extended to other use-cases in which one or more actions need to be decided based on input information, wherein at least one of the actions includes obtaining additional information.

Implementations of the subject matter and the operations described in this specification can be realized in digital electronic circuitry, or in computer software, firmware, or hardware, including the structures disclosed in this specification and their structural equivalents, or in combinations of one or more of them. Implementations of the subject matter described in this specification can be realized using one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

While certain arrangements have been described, the arrangements have been presented by way of example only, and are not intended to limit the scope of protection. The inventive concepts described herein may be implemented in a variety of other forms. In addition, various omissions, substitutions and changes to the specific implementations described herein may be made without departing from the scope of protection defined in the following claims.

1. A computer-implemented method for training a neural network to determine a triage level for a patient, the neural network being for controlling an agent to determine a triage class for one or more patients, the method comprising:
requesting information regarding one or more observed attributes of a patient;
receiving the information regarding the one or more observed attributes of the patient;
defining a state of the patient for an initial step, the state comprising the one or more observed attributes;
for one or more steps beginning at the initial step and continuing until either a triage action is selected or a maximum number of steps has been reached:
inputting the state for the step into the neural network to determine an action in response to the input state, the action being selected from a plurality of actions including: an information request action, and a plurality of triage actions;
obtaining a reward for each of the triage actions based on a probability of the corresponding triage action being correct that is determined based on a set of predetermined triage actions for the patient; and
applying the selected action, including:
in response to the information request action being selected, requesting one or more additional observed attributes of the patient and, in response to receiving the one or more additional observed attributes:
defining a next state for a next step to include the one or more observed attributes and the one or more additional observed attributes of the patient;
defining an experience for the step comprising the state for the step, the selected action, a reward for each triage action of the predefined selection, and the next state; and
moving to the next step; and
in response to one of the triage actions being selected, assigning a corresponding triage level for the selected triage action to the patient; and
updating parameters of the neural network based on the defined experiences.

2. The method of claim 1 wherein:
the neural network is configured to determine state-action values for each action based on input states and the parameters of the neural network;
the actions are determined based on the state-action values for the actions; and
updating parameters of the neural network based on the defined experiences comprises:
selecting a set of one or more experiences from the defined experiences and, for each experience in the set of one or more experiences:
determining, for each of the plurality of actions, a state-action value for the state in the experience using the neural network;
calculating, for each of the plurality of actions, a target state-action value for the state in the experience; and
determining, for each of the plurality of actions, a difference between the corresponding state-action value and the corresponding target state-action value; and
updating the parameters of the neural network based on each determined difference.

3. The method of claim 2 wherein calculating, for each of the plurality of actions, a target state-action value for the state in the experience comprises:
determining the target state-action value for each of the triage actions based on the reward for the corresponding triage action; and
determining the target state-action value for the information request action based on the target state-action values for the triage actions.

4. The method of claim 3 wherein the target state-action value for the information request is determined based on a maximum state-action value of the state-action values either for the triage actions or for a predetermined selection of the triage actions.

5. The method of claim 4 wherein determining the target state-action value for the information request comprises determining a probability that the maximum state-action value for the state relates to a triage action that is incorrect given the state.

6. The method of claim 4 wherein determining the target state-action value for the information request comprises determining a probability that either the maximum state-action value for the state relates to a triage action that is incorrect given the state or that a maximum state-action value for the next state relates to a triage action that is correct given the next state.

7. The method of claim 4 wherein determining the target state-action value for the information request comprises determining a probability that a maximum state-action value for a subsequent state relates to a triage action that would be correct given the subsequent state and that the maximum state-action value for each state before the subsequent state relates to a triage action that is incorrect given the corresponding state.

8. The method of claim 1 further comprising determining the reward for each triage action, including calculating, for each triage action, a probability that the corresponding triage action is correct based on the set of predetermined triage actions for the corresponding patient.

9. The method of claim 8 wherein the probability that the corresponding triage action is correct is determined based on a number of times the corresponding triage action appears in the set of predetermined triage actions for the corresponding patient.

10. The method of claim 1 wherein applying the neural network comprises obtaining a set of observed attributes of the patient, wherein defining the state includes selecting a subset of the set of observed attributes of the patient, and the one or more additional observed attributes are obtained through selection from the set of observed attributes.

11. The method of claim 1 wherein:
the neural network is configured to determine state-action values for each action based on input states and the parameters of the neural network;
the actions are determined based on the state-action values for the actions; and
wherein noise is added to the state-action value for the information request action prior the determination of the action.

12. The method of claim 11 wherein noise is only added to the state-action value for the information request action.

13. The method of claim 11 wherein the noise decays over the number of steps.

14. A computer-implemented method for determining a triage level for a patient including:
obtaining a neural network trained according to the method of claim 1;
requesting information regarding one or more observed attributes of the patient;
receiving the information regarding the one or more observed attributes of the patient;
defining a state of the patient for an initial step, the state comprising the one or more observed attributes;
for one or more steps beginning at the initial step and continuing until either a triage action is selected or a maximum number of steps has been reached:
inputting the state for the step into the neural network to determine an action in response to the input state, the action being selected from a plurality of actions including: an information request action, and a plurality of triage actions;
applying the selected action, including:
in response to the information request action being selected, requesting and receiving information regarding one or more additional observed attributes of the patient, defining a next state for a next step to include the one or more observed attributes and the one or more additional observed attributes of the patient and moving to the next step: and
in response to one of the triage actions being selected, outputting the corresponding triage level for the selected triage action as the triage level for the patient.

15. A system comprising a processor configured to perform the method of claim 1.

16. A system comprising a processor configured to perform the method of claim 14.

17. A non-transitory computer readable medium having stored therein computer executable instructions that, when executed by a processor, cause the processor to perform the method of claim 1.

18. A non-transitory computer readable medium having stored therein computer executable instructions that, when executed by a processor, cause the processor to perform the method of claim 14.
